(19) United States
(12) Patent Application Publication
(10) Pub. No.: US 2019/0362852 A1
(43) Pub. Date: Nov. 28, 2019

(54) MEDICAL VIEWING CERTIFICATES FOR MOBILE DEVICES

(71) Applicant: KONINKLIJKE PHILIPS N.V., Eindhoven (NL)

(72) Inventors: JENS VON BERG, HAMBURG (DE); RAVINDRA BHAT, EINDHOVEN (NL); ANAND KUMAR DOKANIA, UTRECHT (NL)

(73) Assignee: KONINKLIJKE PHILIPS N.V., EINDHOVEN (NL)

(21) Appl. No.: 16/472,363

(22) PCT Filed: Dec. 22, 2017
(86) PCT No.: PCT/EP2017/084499

§ 371 Date: Jun. 21, 2019

(30) Foreign Application Priority Data
Dec. 22, 2016 (IN) 201641043897
Jan. 12, 2017 (EP) 17151104.1

(51) Int. Cl. G16H 50/30; G16H 40/20; G16H 40/40; G16H 30/20

(52) U.S. Cl. CPC G16H 50/30; G16H 40/20; G16H 80/00; G16H 30/20; G16H 40/40

(57) ABSTRACT

In modern health systems, and particularly in those in developing countries, primary care-givers located remotely from diagnosticians are increasingly turning to the possibilities afforded by social media and improved communication techniques. In particular, medical information may be obtained from a patient of the primary care-giver, and transmitted (for example, by email) to a diagnostician. However, the primary care giver has no way to know that the diagnostician has diagnostic information display equipment which could enable a secure diagnosis, for example. The present application discusses diagnosis authentication techniques. The primary care-giver's operator terminal (20) may challenge the diagnostic terminal (10) to certify and transmit receive various attributes useful for assuring a reasonable diagnosis.

### FIELD OF THE INVENTION

The invention relates to a diagnostic terminal for providing suitability certificate data of a user of the diagnostic terminal, to an operator terminal. The invention also relates to an operator terminal, a method for operating a diagnostic terminal, a method for operating an operator terminal, a distributed medical information assurance system, a distributed medical information assurance method, a computer program element for controlling a diagnostic terminal and/or an operator terminal, and a computer-readable medium.

### BACKGROUND OF THE INVENTION

The modern ubiquity of social media and mobile devices can also be observed in the medical profession. It is now possible for a primary caregiver to obtain images from a patient, and to send the images to peers, or to subject-matter specialists. This enables a more complete diagnosis, or a second opinion, to be sought for a certain medical image. Even though this may not always be strictly in accordance with medical guidelines, it is informally experienced in many countries. In areas without access to formal health systems, the quality of care can be increased with access to such informal networks.

The patent application US 2013/0110527 A1 discusses a dental consultation system including a network, a computerized tomography (CT) scanner for obtaining CT digital image file and pictures of the teeth of an oral cavity of a patient. A patient-end terminal is provided for in the input of the CT digital image file and pictures of a patient which were obtained from the CT scanner and the consultation data. This system involves the transmission of certified dentist data including dentist certification, dentist educational program, and purchase of dental medical materials by the dentist member. However, such systems can be further improved.

### SUMMARY OF THE INVENTION

According to a first aspect of the invention, there is provided a diagnostic terminal for providing suitability certificate data of a user of the diagnostic terminal, to an operator terminal, comprising:

a first data communication interface;

a first display;

a first input interface; and

a first processing unit.

The first processing unit is configured to generate suitability assessment response data based on a suitability test executed by the diagnostic terminal and by the user of the diagnostic terminal, to generate suitability certificate data based on the suitability assessment response data generated by the diagnostic terminal, and to transmit the suitability certificate data and/or the suitability assessment response data to an operator terminal via the first data communication interface.

Accordingly, it is possible for a diagnostician situated remotely from a local caretaker to generate a verification that the diagnostician is able to perform diagnoses at an adequate quality level with the equipment used to make the diagnosis. A suitability certificate can be generated based on a number of tests. The suitability certificate, along with the test response information, can be communicated to a local caretaker using an operator terminal. This improves the trust between the local caretaker and the diagnostician. In addition, the otherwise inconvenient process of verifying that the diagnostician is able to make accurate diagnoses using their equipment is made much faster and easier according to the first aspect.

Optionally, the diagnostic terminal according to the first aspect generates the suitability assessment response data based on intrinsic data of the diagnostic terminal, and/or a suitability test executed by the diagnostic terminal.

Optionally, a diagnostic terminal is provided according to the first aspect, wherein the first processing unit is further configured to receive suitability assessment challenge request data transmitted to the diagnostic terminal by a remotely located operator terminal. The suitability assessment response data is generated based on the requirements of the suitability assessment challenge request data. The suitability certificate data and/or the suitability assessment response data is transmitted to the operator terminal via the first data communication interface, as a response to the suitability assessment challenge request data.

Accordingly, a local caretaker can transmit, from an operator terminal, a suitability assessment challenge request, which proactively requests that the diagnostician undergoes a series of tests (or his equipment) in order to establish a trusted connection in a simple way.

Optionally, a diagnostic terminal is provided, wherein the first processing unit is configured to generate the suitability assessment response data by interrogating the diagnostic terminal to derive a display contrast characteristic of the first display of the diagnostic terminal.

Accordingly, information about the display contrast and/or quality may be provided automatically, based on device settings or parameters that are already held inside the diagnostic terminal and are associated with the display of the diagnostic terminal. In this embodiment, the diagnostician does not need to provide, or participate in a test because the display contrast characteristic is obtained automatically.

Optionally, the diagnostic terminal is provided with a camera.

The first processing unit is configured to generate the suitability assessment response data by displaying a contrast test sequence on the first display of the diagnostic terminal, to record the contrast test sequence generated by the first display of the diagnostic terminal using the camera, wherein the camera and the display of the diagnostic terminal are arranged to face a mirror simultaneously, and to generate the suitability assessment response data by comparing the recorded contrast test sequence to a set of standard contrast test sequence results wherein the suitability assessment response data comprises diagnostic terminal display contrast data of the first display.

Accordingly, a front-facing camera of, for example, a smartphone, as would be used for internet conference calling, can be directed at a mirror. A contrast test image or sequence can be replayed by the diagnostic terminal's first display, and simultaneously the contrast test image or sequence is recorded by the camera. This is a way to simply, but accurately, record the display capability of the diagnostic terminal, to ensure that it is suitable for the display of medical images.

Optionally, the processing unit of the diagnostic terminal is configured to generate the suitability assessment response data by displaying, on the first display of the diagnostic terminal, a certified graphical test image, or a certified video test sequence. The diagnostic terminal is considered to receive, via the first input interface of the diagnostic terminal, a certified graphical test or a certified video selection as a user input command, and to generate the suitability assessment response data by comparing the certified graphical test, or the certified video selection, to a set of graphical tests or a set of video selection feedback results. The first processing unit is configured to provide the suitability assessment response data comprising the certified graphical test or the certified video selection of the first display.

Accordingly, the diagnostic terminal may be used to provide a certified test image, or a certified video sequence which can be used to assess the visual ability of a diagnostician in combination with a certain diagnostic terminal display unit. Because the test or videos are certified by test software in the diagnostic terminal, and/or the operator terminal, it is difficult to defeat such a test.

Optionally, the processing unit of the diagnostic terminal is configured to generate the suitability assessment response data by commanding the diagnostic terminal to yield display configuration information of the first display, to verify the display configuration information, to generate the suitability assessment response data by certifying the verified display configuration data, and wherein the suitability assessment response data comprises the certified display configuration data.

Accordingly, display configuration information, such as display size, and display resolution, may be reported back to the operator terminal. This enables the operator terminal to be assured that the field of view of the display is satisfactory.

Optionally, the processing unit of the diagnostic terminal is configured to generate the suitability assessment response data by displaying, on the display of the diagnostic terminal, a plurality of medical test images and/or videos, and for each of the displayed images and/or videos in the plurality of medical test images and/or videos, the first processing unit is configured to present a challenge input prompt to a user on the first display of the diagnostic terminal and to obtain a set of challenge answers via the first input interface, to analyze the set of challenge answers, to generate an image and/or video diagnostic capability level, and to generate the suitability assessment response data by certifying the image and/or video diagnostic capability level, wherein the suitability assessment response comprises the certified image level and/or the certified video diagnostic level.

Accordingly, a series of typical detection tasks in radiology may be provided to a diagnostician using the diagnostic terminal. These images can be used as a test, to test the ability of the diagnostician to identify relevant radiological science based on the display quality and the vision and expertise of the diagnostician.

Optionally, the suitability certificate data comprises, or is, an encrypted data portion encrypted by the first processing unit.

Accordingly, it is possible to ensure that the test of the diagnostic terminal has been authentically generated.

Optionally the suitability certificate data comprises an authenticated time-stamp representing a time point when the suitability assessment response data was generated. Accordingly, it is possible to test the age of a diagnostician's test data, or suitability certificate data.

Optionally, the first processing unit is further configured to calculate a time-delay of an authenticated time-stamp of the suitability certificate data from a current time point, and wherein, if the time lapse is greater than a certification time threshold, the first processing unit is configured to repeat the generation of the suitability certificate data at the diagnostic terminal.

Accordingly, the diagnostic terminal is able to regenerate a suitability certificate if the suitability certificate is too old.

Optionally the processing unit is further configured to receive (i) diagnosis request data and (ii) pre-diagnostic data via the first data communication interface from an operator terminal. The first processing unit is configured to display the diagnosis request data and the pre-diagnostic data to a medical professional, to receive diagnosis data from the medical professional, to generate certified diagnosis data by combining the suitability certificate data with the diagnosis data, and to transmit the certified diagnosis data to an operator terminal via the first data communication interface.

Accordingly, a pre-diagnostic data, such as an undiagnosed image of a lung infection, can be diagnosed by a medical professional using the diagnostic terminal. The diagnosis data, such as a photograph or an X-ray of a patient received from an operator terminal, may have a certified diagnosis data item added to the diagnosis data to enable a local caregiver to understand the diagnostic competence of the diagnostician at first side. For example, a quality code could be embedded in the image forming the diagnosis data.

According to a second aspect, there is provided a method for operating a diagnostic terminal to provide a suitability certificate data of a user of the diagnostic terminal, to an operator terminal, comprising:
a) generating a suitability assessment response data based on or a suitability test executed by the diagnostic terminal and/or by the user of the diagnostic terminal;
b) generating suitability certificate data based on the suitability assessment response data generated by the diagnostic terminal; and
c) generating the suitability certificate data and/or the suitability assessment response data to an operator terminal via a data communication interface.

According to a third aspect of the invention, there is provided an operator terminal for acquiring suitability assessment response data and/or suitability certificate data from a user of the diagnostic terminal. The operator terminal comprises:

a second data communication interface;

a second display;

a second input interface; and

a second processing unit.

The second processing unit is configured to receive suitability certificate data based on a suitability assessment result generated at the remote diagnostic terminal from the second data communication interface, to analyze the suitability certificate to provide a diagnostic terminal suitability result, and to display the suitability certificate, and/or the diagnostic terminal suitability result to a user of the operator terminal using the second display.

Accordingly, a local caregiver may use the operator terminal to verify that a remote diagnostician using a remote diagnostic terminal is using a suitable terminal, and has the medical skill, to diagnose a certain type of image or medical condition.

Optionally, the operator terminal is provided wherein the second processing unit is further configured to transmit suitability assessment challenge request data to a remote diagnostic terminal via the second data communication interface, and to instruct the remote diagnostic terminal to generate suitability certificate data and/or suitability assessment response data.

Accordingly, a local caregiver may proactively interrogate a remote diagnostic terminal to receive assurances about the suitability of the diagnostic terminal, or as user, in diagnosing medical images.

Optionally, the second processing unit is further configured to analyze the suitability certificate data by identifying an encrypted data portion of the suitability certificate, decrypting the encrypted data portion of the suitability certificate, generating a diagnostic trust metric based on the decrypted result of the encrypted data portion of the suitability certificate, and displaying the diagnostic trust metric to a user of the operator terminal using the second display.

Accordingly, the local caregiver can verify with certainty that the remote diagnostician has generated the suitability certificate using a valid version of a diagnostic terminal test software application.

Optionally, the second processing unit is further configured to analyze the suitability certificate data by extracting results of one, or a combination, of a diagnostic terminal contrast response, a graphical test or a video selection feedback result, certified display configuration data, or a certified image level and/or a certified video diagnostic level, to compare at least the results of one, or a combination of, a diagnostic terminal contrast response, a graphical test or a video selection feedback result, certified display configuration data, or a certified image level and/or a certified video diagnostic level with standard values, and to generate a diagnostic terminal test threshold, and wherein the second processing unit is configured to display the diagnostic terminal test threshold to a user on the second display.

Accordingly, one, or a variety of tests may be used to verify the ability of a remote diagnostic terminal, and/or a remote diagnostician, to accurately fulfil the requirements of a particular medical test.

Optionally, the second processing unit is further configured to extract, from the suitability certificate data, an authenticated time-stamp representing the time point that the suitability assessment challenge had been performed, and to display, to a user of the operator terminal, the time point that the suitability assessment challenge had been performed using the second display.

Accordingly, the local caregiver using the operator terminal is able to assess how old the suitability certificate data is before proceeding with a diagnostic exchange with the local diagnostician.

Optionally, the processing unit is further configured to transmit to a diagnostic terminal (i) a diagnosis request and (ii) pre-diagnostic information via the second data communication interface, to receive, from the diagnostic terminal via the second data communication interface, certified diagnosis data comprising diagnosis data and a suitability certificate, and to display, to a user of the operator terminal, the diagnosis data and the suitability certificate using the second display.

According to a fourth aspect, there is provided a method of operating an operator terminal to acquire suitability assessment response data and/or suitability certificate data of a user of the diagnostic terminal. The method comprises:
b1) receiving suitability certificate data based on a suitability assessment result generated at the remote diagnostic terminal;
b2) analyzing the suitability certificate to provide the diagnostic terminal suitability result; and
b3) displaying the suitability certificate, and/or the diagnostic terminal suitability result to a user of the operator terminal.

According to a fifth aspect of the invention, there is provided a distributed medical information assurance system. The system comprises: a diagnostic terminal as described according to the first aspect or one of its embodiments, and an operator terminal according to the third aspect or one of its embodiments.

According to a sixth aspect, there is provided a distributed medical information assurance method comprising:
p) generating suitability assessment response data based on a suitability test executed by the diagnostic terminal and/or by the user of the diagnostic terminal;
q) generating suitability certificate data based on the suitability assessment response data generated by the diagnostic terminal;
r) transmitting the suitability certificate data and/or the suitability assessment response data to an operator terminal via data communication interface;
s) receiving suitability certificate data based on suitability assessment result generated of the remote diagnostic terminal;
t) analyzing the suitability certificate to provide a diagnostic terminal suitability result; and
u) displaying the suitability certificate, and/or the diagnostic terminal suitability result to a user of the operator terminal.

According to a seventh aspect there is provided a computer program element for controlling the diagnostic method according to the first aspect or its embodiments, and/or an operator terminal according to the third aspect and its embodiments, which, when the computer program element is executed by a processing unit, is adapted to perform the method of the second or fourth aspect, respectively.

According to an eighth aspect of the invention, there is provided a computer-readable medium having stored the computer program element of the seventh aspect. It is, thus, a basic idea of the invention to enable a local caregiver to exchange diagnostic data with a diagnostician, using remote terminals having an enhanced level of trust between them. The enhanced level of trust is generated by the fulfilment of the diagnostic terminal, and/or diagnostician, by one, or a number of tests.

Some of tests require only the evaluation of technical details of the diagnostician's device, and others of the test require a qualitative test of the diagnostician's abilities. The suitability assessment response data may thus be based solely on configuration data held intrinsically on the diagnostic terminal, on data input by a diagnostician into the diagnostic terminal, or on a mixture of both. Of course, the suitability assessment response data may be generated proactively by the diagnostic terminal, optionally after a time delay, to enable one, or many local caregivers to interact with the diagnostician quickly. Alternatively, a local caregiver may proactively challenge the diagnostician by sending a suitability assessment challenge request to the diagnostic terminal.

In the following description, the term “suitability certificate data” means a data structure representing an authenticated assessment indicative of the ability of a diagnostic terminal, or the user of that terminal, to form a valid opinion about a medical diagnostic situation, typically the display of an image such as a radiographic image to the user of the diagnostic terminal. The suitability certificate data may be generated as the end result of one, or a series, of suitability assessment tests operated at the diagnostic terminal. In a first alternative, the suitability certificate data may be proactively generated by a diagnostic terminal and contain, for example, a resolution limit of the diagnostic terminal. Upon challenge by an operator terminal, the suitability certificate data, comprising a certified resolution of the diagnostic terminal, may be transmitted to the operator terminal. In this case, the user of the operator terminal would know from the certified resolution of the diagnostic terminal contained in the suitability certificate data, that only images of a certain resolution could be examined by the user of the diagnostic terminal. Optionally, the suitability certificate data may be encrypted at the diagnostic terminal to ensure the privacy of the diagnostician's diagnostic ability.

Optionally, the suitability certificate data comprises a series of results for one or more of the suitability assessment tests. Optionally, the suitability certificate data may contain an authentication field containing a public and/or private cryptographic key. In the following description, the term “suitability assessment response data” means initial data gathered at the diagnostic terminal during a test or challenge applied at the diagnostic terminal. In the case of an assessment of screen contrast of a diagnostic terminal, the suitability assessment response data may be a contrast response curve of the display of the diagnostic terminal.

Optionally, the suitability assessment response data may be converted into suitability certificate data at the diagnostic terminal, using certified software installed on the diagnostic terminal. Then, only the suitability certificate data is transmitted onwards to the operator terminal. This means that the amount of data that needs to be transmitted between the diagnostic terminal and an operator terminal is reduced to only the suitability certificate data.

Alternatively, the suitability assessment response data may be transmitted with the suitability certificate data to the operator terminal. This allows the operator terminal to certify separately the suitability assessment response data at the operator terminal, as a double-check of the diagnostic ability of the diagnostic terminal, and/or the user of the diagnostic terminal.

In the following application, the term “intrinsic data” refers to set-up parameters, configuration parameters, and other settings which are native to the components of the diagnostic terminal. For example, an example of intrinsic data is the resolution of the display of the diagnostic terminal, for example 1024 by 768 pixels. Such intrinsic data may be stored on firmware, hardware, or in the application environment (operating system) of the diagnostic terminal and be accessible for interrogation by the application environment of the diagnostic terminal.

In the following description, the term “suitability test” refers to an information gathering process coordinated by a processing unit of the diagnostic terminal which enables data structures representing the suitability assessment response data to be assembled. In other words, the suitability test may, in a simple form, be a “look-up” of the intrinsic data of the diagnostic terminal using the application environment. Alternatively, the suitability test may be a series of challenges presented to a user of the diagnostic terminal presented by a display of the diagnostic terminal, and an input interface of the diagnostic terminal.

In the following description, the term “suitability assessment challenge request” refers to a request, transmitted to a diagnostic terminal from an operator terminal, to provide information about the ability of a diagnostic terminal, or the user of the diagnostic terminal. Satisfaction of the suitability assessment challenge request enables a caregiver at the operator terminal to trust medical assessments made at the diagnostic terminal satisfactorily. For example, the operator may wish to know the ability of the diagnostic terminal to read a high resolution radiographic image. Thus, a suitability assessment challenge request would be transmitted to the diagnostic terminal requesting information about the resolution of the diagnostic terminal's display. In this case, the resolution of the display represents suitability assessment response data. In one option, the diagnostic terminal may receive, as part of the suitability assessment challenge request, a resolution requirement. Certified software operating on the diagnostic terminal may compare the required resolution contained in the suitability assessment challenge request with the resolution of the display of the diagnostic terminal and issue a suitability certificate to the operator terminal with a simple binary “1” or “0” representing a yes or no response to the suitability assessment challenge request. Alternatively, the suitability certificate data may be transmitted alongside the resolution (suitability assessment response data) to the operator terminal, so that the operator terminal may make the final judgement about whether or not the diagnostic terminal is able to satisfactorily assess the images, video sequence, or sound.

### BRIEF DESCRIPTION OF THE DRAWINGS

Exemplary embodiments will be described with reference to the following drawings:

FIG. 1a) shows a diagnostic terminal according to an aspect of the invention.

FIG. 1b) shows an operator terminal according to an aspect of the invention.

FIG. 2) shows an example of a diagnostic terminal during an assessment test)

FIG. 3 shows an example of a diagnostic terminal test image as a collection of Landolt rings of different sizes.

FIGS. 4a) to 4g) show optional examples of data packet formats.

FIG. 5 shows a distributed medical information assurance system.

FIG. 6a) shows a diagnostic terminal method.

FIG. 6b) shows an operator terminal method.

FIG. 7 shows a distributed medical information assurance method.

### DETAILED DESCRIPTION OF EMBODIMENTS

The improvement in communication brought by modern social networks and communication devices means that primary caregivers can transfer medical information to a remote diagnostician. When users are operating a mobile diagnostic device (such as a portable X-ray scanner, ultrasound scanner, or electronic stethoscope) at a location where no diagnostic expertise is available, a primary caregiver may be educated to operate a mobile diagnostic device. The primary caregiver may not be able to achieve a secure diagnosis from the information obtained at the care location, and may prefer to seek a second opinion. The local caregiver could be operating an imaging device designed for radiographical purposes, in the case of an emergency, for example

In such a situation, the local caregiver would feel a responsibility for their local patient. Trusting a remotely given diagnosis may be an issue. The local caregiver needs to be able to trust the diagnosis, or at least to know the limits that they can place on the remote diagnosis.

The quality of a remote diagnosis can be verified, for example, by finding out the technical ability (such as a display resolution) of a device used to assess medical information transmitted from the diagnostic device An assurance that a specific medical diagnosis, or follow-up advice, has been based on reading diagnostic information which has been obtained with a well-defined quality will be desirable, even if it is generally accepted that devices like mobile phones and tablets may be used to help with the remote diagnosis.

Still further, a local caregiver may desire information about the relative ability of a local diagnostician to provide a particular diagnosis. In this case, the local caregiver may challenge a remote diagnostician with a series of tests which verify the diagnostician's ability to provide a reliable diagnosis. The provision of such suitability certificate data generated and transmitted by a diagnostic terminal is also in the interest of the person providing the diagnosis. Otherwise, the quality of the diagnostician's advice or diagnosis could be questioned by the local caregiver. Therefore, a suitability assessment test may be provided to test the technical ability of a diagnostic terminal, and/or the diagnostic ability of a remote diagnostician. Once the test has been passed successfully, a secure certificate is generated on the remote diagnostic terminal, which can be sent automatically with a diagnosis.

In the following application, several tests are envisaged which will briefly be summarized for context.

Optionally, a display contrast test enables a local caregiver to be certain about the ability of a remote diagnostic terminal to display an image with sufficient contrast. Optionally, a resolution test may be used to ensure that a diagnostician using the diagnostic terminal can read generic symbols on a display screen of the diagnostic terminal to a minimally detectable size.

Optionally, a field of view test may be applied to ensure that a display of a remote diagnostic terminal has a sufficient display resolution, for example, to display a radiographic image in one piece, without requiring extensive scrolling around a diagnostic image.

A further test optionally tests the ability of the remote diagnostic terminal, and its user, to detect a series of medical pathologies in test medical images. Of course, this test may be extended to test the ability of a medical professional to detect sequences of a video accurately, for example an ultrasound video sequence.

Sound is also an important feature when making a diagnosis, and is useful when detecting heart murmurs or breathing problems, using an electronic stethoscope, for example.

Therefore, the ability of an audio generating means of a remote diagnostic terminal, for example a loudspeaker or headphones, to accurately enable the reproduction of the sound recorded or input into an operator terminal can be tested.

It will be appreciated that the approach detailed herein is divided between an operator terminal operated by a local caregiver, and a diagnostic terminal operated by a remote diagnostician. These devices may optionally be used as part of a medical assurance system. It will also be appreciated that a variety of encryption and authentication measures may optionally be taken to ensure that the suitability assessment response data and the suitability certificate data has not been tampered with at the diagnostic terminal end, or the operator terminal end. Such measures can provide the local caregiver with improved assurance about the correctness of a remote diagnosis, and such measures can also provide the remote diagnostician with assurance that a diagnosis will not be misused at the local caregiver side.

In the following description, the attributes of the diagnostic terminal and the test which it can perform are discussed first. Then, the operator terminal and its interaction with the diagnostic terminal is discussed. Finally, the disposition of the diagnostic terminal and the operator terminal as part of a system is discussed.

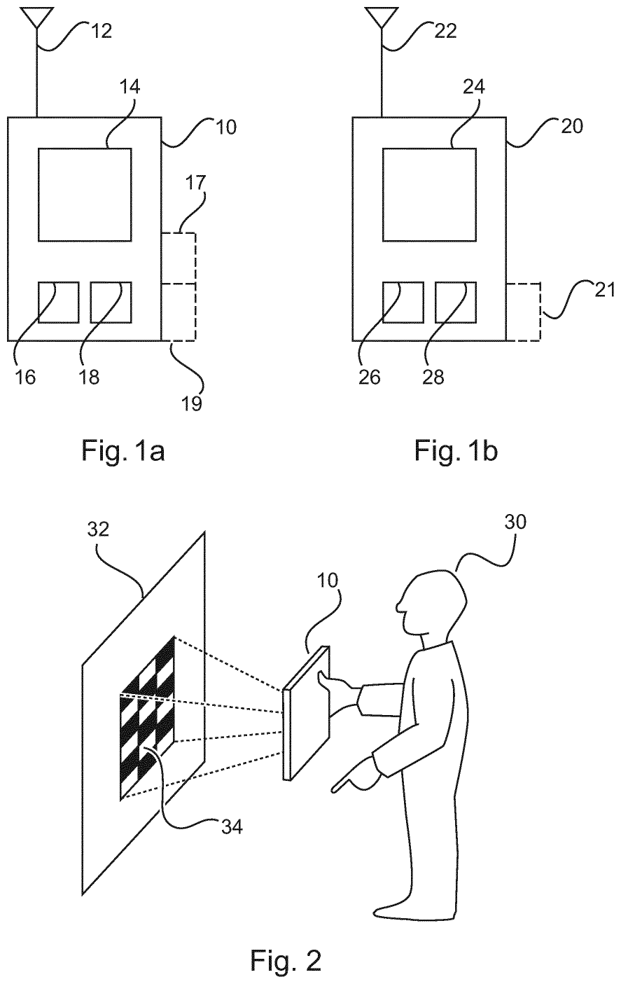

FIG. 1a) shows a diagnostic terminal 10 for providing suitability certificate data of the diagnostic terminal, and/or a user of the diagnostic terminal, to an operator terminal 20. The diagnostic terminal 10 typically comprises a first data communication interface 12, a first display 14, a first input interface 16, and a first processing unit 18. As will be appreciated by a person skilled in the art, these electronic components may be coupled to power supplies, bus communication interfaces, memories, and the like to enable their functional operation.

The first data communication interface 12 enables the communication of data between the diagnostic terminal 10 and an external data network, or other data communication device. For example, the first data communication interface 12 may comprise a “Wi-Fi”(™) modem, operatively coupled to the first processing unit 18 of the diagnostic terminal 10. Thus, a diagnostic terminal 10 can communicate with other devices using a LAN, or WAN connection, for example to the internet. Alternatively, the first data communication interface 12 may comprise an Ethernet connection, a Bluetooth (TM) connection, a GSM(™), 3G, 4G, or a 5G data connection.

The first display 14 of the diagnostic terminal 10 is operatively coupled to the first processing unit 18. Therefore, diagnostic information received by the diagnostic terminal via the first data communication interface 12, for example, may be displayed on the first display 14 by the first processing unit 18.

A software program executed natively on the first processing unit 18 of the diagnostic terminal 10 may be loaded into operating memory of the diagnostic terminal 10, so that it displays a Graphical User Interface (GUI) on the first display 14. The first display unit 14 has attributes such as horizontal and vertical resolution, contrast ratio, color depth, refresh rates, and many other attributes which affect the quality of displayed images. Optionally, the first display comprises firmware holding information on these display-specific attributes. Optionally, the display-specific attributes (examples of intrinsic data) may be held on a memory of the diagnostic terminal 10, for example, as part of the operating system software of the diagnostic terminal 10.

The diagnostic terminal 10 further comprises a first input interface 16. The first input interface 16 enables a user of the diagnostic terminal 10 to control images that are displayed on the first display 14, for example. Therefore, in an example, a sequence of images may be displayed to a user of the diagnostic terminal 10 using the first display 14, and the user of the diagnostic terminal 10 may answer “Yes” or “No” questions about images of the sequence of images displayed on the first display 14. The first input interface 16 would optionally be implemented as one or more of: a keyboard, a touchpad, or a mouse. Optionally, the first input interface 16 is a touch-screen display integrated with the first display 14.

Optionally, the diagnostic terminal 10 comprises a first audio interface 19.

This may be implemented as a loudspeaker, or an audio output port (suitable for connection to headphones, for example) of the diagnostic terminal 10. The first audio interface 19 may be capable of reproducing sounds such as the output of an electronic stethoscope. Thus, optionally a first audio interface 19 of the diagnostic terminal 10 is also operatively coupled to the first processing unit, the first input interface, and the first data communication interface.

The first processing unit 18 is operatively coupled to the first input interface 16, the first display 14, and the first data communication interface 12. The first processing unit 18 executes an operating system of the diagnostic terminal 10, and generally coordinates the communication of information to and from the diagnostic terminal 10 using the first data communication interface 12, enables the display of diagnostic challenges using the first display 14, and enables a response to the diagnostic challenges to be recorded using the first input interface 16. The first processing unit 18 may optionally be implemented using, for example, a microprocessor, or other processor incorporating for example, an ARM(™) processor core, or other processor.

The skilled person will appreciate that a typical diagnostic terminal 10 falling within the above general description may comprise, for example, a smartphone, or a tablet computing device, a laptop, a personal computer, or the like. According to a first aspect, there is provided a diagnostic terminal 10 for providing suitability certificate data of a user of the diagnostic terminal, to an operator terminal, comprising:

a first data communication interface 12;

a first display 14;

a first data input interface 16; and

a first processing unit 18.

The first processing unit 18 is configured to generate suitability assessment response data based on a suitability test executed by the diagnostic terminal and/or by the user of the diagnostic terminal, to generate suitability certificate data based on the suitability assessment response data generated by the diagnostic terminal, and to transmit the suitability certificate data and/or the suitability assessment response data to an operator terminal via the first data communication interface.

Accordingly, it is possible for the diagnostic terminal 10, and/or a diagnostician using the diagnostic terminal 10, to verify the ability of the diagnostic terminal and/or diagnostician to provide a medical diagnosis of transmitted diagnostic information to a required level.

Optionally, the diagnostic terminal 10 is configured, via the first processing unit 18, to receive a suitability assessment challenge request transmitted to the diagnostic terminal by a remotely located operator terminal 22. For example, the suitability assessment challenge request may comprise a simple challenge requesting the diagnostic terminal 10 to respond with its display resolution. The suitability assessment challenge request may optionally be a request to display a sequence of test images, videos, or sounds to the diagnostician, and to input test results into the diagnostic terminal for transmission back to the operator terminal.

The suitability assessment response data is generated based on the requirements of the suitability assessment challenge request data; and the suitability certificate data and/or the suitability assessment response data is transmitted to the operator terminal via the data communication interface 12 as a response to the suitability assessment challenge request data.

Accordingly, the local caregiver may proactively challenge a remote diagnostician to provide a certified account of the status of their diagnostic terminal 10, or their current ability to perform a diagnosis.

The diagnostic terminal 10 may perform a device contrast check.

Optionally, the diagnostic terminal 10 comprises a camera 17 capable of acquiring individual images, or a video sequence. Typically, the camera will be configured to record an image in the same direction that a display of the diagnostic terminal 10 is facing. In other words, on a typical smartphone, the camera 17 will be the video-conferencing camera (“webcam”) of the smartphone, or tablet device. When performing the self-sufficient device contrast check, the camera 17 is pointed towards a mirror, and a contrast test is run on the first display 14 using certified test software executed by the first processing unit 18 on the first diagnostic terminal 10.

Test images generated by the first processing unit 18 and displayed on the first display 14 vary, for example, the brightness of display areas of the first display 14. The camera 17 of the diagnostic terminal 10 records the generated images in the mirror. This enables the first processing unit 18 to generate, for example, a contrast response curve of the first display 14. Having generated the contrast response curve, the contrast response curve may be provided as suitability assessment response data.

The suitability assessment response data comprising the contrast response curve may be appended to a generated suitability certificate generated by the first processing unit 18.

Optionally, a suitability assessment challenge request may be received from an operator terminal indicating a contrast response requirement. If the contrast response curve forming the suitability assessment response data meets or exceeds the requirement of the operator terminal, the suitability certificate data may contain this information and be transmitted on its own.

Alternatively, the suitability assessment response data may be transmitted as an appendix to the suitability certificate data. This enables the operator terminal to assess the suitability assessment response data (for example, the contrast response curve) independently of the diagnostic terminal 10. In this case, the suitability certificate data provides an authenticated verification that the test was, in fact, run on the first display 14 of the diagnostic terminal 10.

Optionally, the first processing unit 18 is configured to generate the suitability assessment response data by displaying a contrast test sequence on the first display 14 of the diagnostic terminal 10, a certified graphical test image or certified video test sequence, receiving, via the first input interface 16 of the diagnostic terminal 10, a certified graphical test or a certified video selection as a user input command, generating the suitability assessment response data by comparing the certified graphical test, or the certified video selection, to a graphical test or a set of video selection feedback results, and the suitability assessment response data comprises the certified graphical test, or the certified video selection of the first display 14, or a contrast response curve.

Optionally, the diagnostic terminal 10 may receive certified graphical test images or certified video test sequence from the operator terminal, enabling a fully independent verification of the diagnostic terminal 10 to be made. Alternatively, the certified graphical test images or certified video test images may be held in software on the diagnostic terminal 10, preferably in encrypted form, to enable the graphical tests and/or certified video tests to be performed without needing to have a high bandwidth requirement when transferring the test images.

The data requirement to transfer of the result of a test is typically orders of magnitude smaller than the amount of data required to transfer a test image or test sequence itself.

Optionally, the operator terminal may interrogate a remote diagnostic terminal 10 to derive a display contrast characteristic of the first display 14 of the diagnostic terminal, and transmit this as suitability assessment response data.

Typically, displays, or display drivers associated with an operating system executed by a processing unit, contain information about the display capabilities of a terminal display. In this embodiment, a remote operator terminal may interrogate such a display driver information, for example, and return it to the operator terminal as the suitability assessment response data and/or suitability certificate data. Therefore, it may be possible to provide sufficient information about the suitability of the first display 14 without requiring a mirror-test to take place.

FIG. 2 illustrates the first test discussed above. It is seen that a user 30 is holding the diagnostic terminal 10 facing towards a minor 32. The diagnostic terminal 10 projects a test pattern 34 onto the mirror 32. In FIG. 2, the projected test pattern is derived from a chess board pattern, in which the individual squares of the chess pattern may have different intensities. A camera of the diagnostic terminal 10 (not shown) records the test pattern reflected from the mirror 32, enabling the processing unit 18 of the diagnostic terminal 10 to record and generate suitability assessment response data, and suitability certificate data based on the recorded test pattern. For example, the recorded test pattern may be compared to standard values held in the test software on the diagnostic terminal 10. The number of squares in the checked pattern not meeting required standard values may be reported as suitability assessment response data.

The diagnostic terminal 10 is also configurable to provide a resolution test. This test displays generic test patterns having a differing resolution, for example. The tests are displayed on the first display 14 to the diagnostician. The diagnostician should then indicate whether, or not, they are able to identify the symbols.

Optionally, symbols such as Landolt rings, or the letter “E” in varying orientations may be displayed. Optionally, the software stored on the diagnostic terminal 10 may comprise a series of generic test signs. Alternatively, the operator terminal may transmit display signs to the diagnostic terminal for use. When the display signs are transmitted from the operator terminal, it is easier to ensure the integrity of the test. However, when the display signs are generated at the diagnostic terminal 10, without requiring transmission from the operator terminal, it is easier to perform the suitability assessment over a low-bandwidth communication link. Therefore, the software in the diagnostic terminal 10 and the operator terminal is certified, it is hard for a diagnostician to subvert the test. If the display quality of the first display 14 is not a sufficient accuracy, or if the visual accuracy of the observer is limited, the observer will not be able to identify the signs reliably.

Psychophysical methods allowing for a quick determination of the resolution threshold without the need for testing of all possible combinations of resolution and size of the display patterns allow for a quick determination.

The test need not necessarily start with very large signs, and intelligent test protocol can minimize the number of binary decisions to be taken. The test output is, in one option, a threshold about the minimally detectable size of the display pattern. The minimally detectable size threshold may be generated as suitability assessment response data on the diagnostic terminal 10, and certified with a suitability certificate generated at the diagnostic terminal 10. Then, the minimally detectable size threshold may be transmitted with the suitability certificate to the operator terminal.

Optionally, the raw sequence of test decisions relating to the test patterns may be transmitted to the remote operator terminal as suitability assessment response data, certified with a suitability certificate data, and the minimally detectable size threshold may be derived at the operator terminal. Such an option ensures higher security, because the minimally detectable size threshold is calculated at the operator terminal 20, rather than at the diagnostic terminal 10.

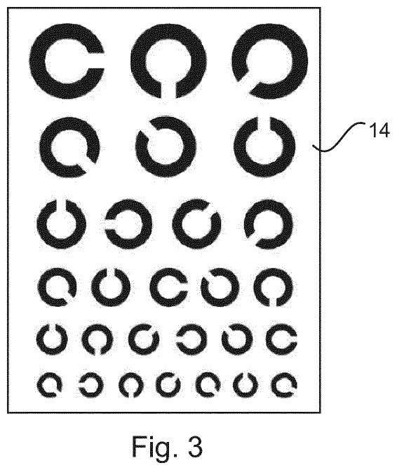

FIG. 3 illustrates a selection of Landolt rings of different sizes that can optionally be used to determine the spatial resolution of the first display 14. It will be appreciated that many test patterns may be used, and suited to specific circumstances. Optionally, the first processing unit 18 is configured to generate the suitability assessment response data by displaying, on the first display 14 of the diagnostic terminal, a certified graphical test image, or certified video test sequence, receiving, via the first input interface 16 of the diagnostic terminal 10, a certified graphical test or a certified video selection as a user input command, generating the suitability assessment response data by comparing the certified graphical test, or the certified video selection, to a set of graphical test or a set of video selection feedback results, and wherein the suitability assessment response data comprises the certified graphical test or the certified video selection of the first display 14.

Optionally, a field of view test is provided by the diagnostic terminal 10. This test assesses the trade-off between display size and display resolution. It provides an assurance to the operator terminal that the first display 14 is able to display a medical image completely, without the need to scroll around an image on an undersized display, for example. The display size, and the number of pixels may already be sufficient information for this, once the resolution test is performed.

For example, the first display 14, or memory inside the diagnostic terminal 10 may comprise information about the display size and number of pixels, comprised in the device driver software of the first display 14.

Alternatively, the data may be held as firmware, or in a hardware element of the first display 14 or the first processing unit 18. The operator terminal 20 may, for example, send a suitability assessment challenge request to the diagnostic terminal 10 requesting the attributes such as display size and number of pixels of the first display 14 to be returned as a suitability assessment response.

Optionally, the first processing unit 18 is configured to generate the suitability assessment response data by commanding the diagnostic terminal to yield display configuration information of the first display 14, to verify the display configuration information, to generate the suitability assessment response data by certifying the verified display configuration data, and wherein the suitability assessment response data comprises the certified display configuration data.

Thus, the certified display configuration data is, for example, a display size, or a number of pixels, held in the firmware, hardware, or memory of the diagnostic terminal 10. Another type of task that can be provided as a suitability test to a diagnostician by the diagnostic terminal 10 is a typical medical detection task, for example, in radiology, which tests the ability of the diagnostic terminal 10, and/or the ability of a user of the diagnostic terminal 10, to identify a diagnostic state of medical test cases, rather than pure vision parameters of the first display 15.

Optionally, the diagnostician is presented with a number of medical test cases to identify findings of increasing clarity. For example, an image may be provided having a number of lung nodules of increasing severity in chest X-ray images of a specimen. Lung nodules, for example, occur in a wide range of shapes. Some of these cases can be taken to test the ability of a reader to identify relevant radiological signs, based on the display quality, and the diagnostician's vision and expertise. The test result, may for example, be a severity threshold to be identified from a lung image.

Examples of other subtle signs to be progressively tested are hair-line fractures in bone images, or so-called “honeycomb patterns” in the lung tissue.

Optionally, the sequence of test images (or test information, such as a test sound) may be provided by the operator terminal 20 to the diagnostic terminal 10. In this case, test images may be selected which are most suitable to the instant detection task of the diagnostician at the diagnostic terminal side 10. This approach also ensures maximum security, because the test information would be delivered to the diagnostic terminal 10 as new information, without the diagnostic terminal 10 having had a chance to compromise the test information.

However, in situations where a low communication bandwidth is present between the operator terminal 20 and the diagnostic terminal 10, it may be more practical to provide a sequence of test-case images in permanent memory of the diagnostic terminal 10. Optionally, these images can be protected via encryption. The operator terminal may optionally transmit a decryption key for a series of test images (or test information such as a test sound) to the diagnostic terminal 10 to enable the detection task test to proceed. In this way, security of the test images may be maintained, or be with a lower communication bandwidth. Alternatively, the decryption key may be sent via a side channel (such as by a ‘phone call or SMS) and entered by the diagnostician into the diagnostic terminal.

Optionally, the first processing unit 18 is configured to generate suitability assessment response data by displaying, on the first display 14 of the diagnostic terminal 10, a plurality of medical test images and/or videos, and for each of the displayed images and/or videos in the plurality of medical test images and/or videos, the first processing unit 18 is configured to present a challenge input prompt to a user of the first display 14 of the diagnostic terminal, and to obtain a set of challenge answers via the first input interface 16 to analyze the set of challenge answers, and to generate an image and/or video diagnostic capability level to generate the suitability assessment response data by certifying the image and/or video diagnostic capability level, wherein the suitability assessment response data comprises the certified image level and/or the certified video diagnostic level.

In other words, the user of the diagnostic terminal 10 may be presented on the first display 14 with one, or a sequence, of test images or videos. The user of the diagnostic terminal 10 may use the input interface 16 to denote one, or a selection, of medical pathologies present in the displayed medical test image or video. After one, or a sequence of medical test images and/or videos have been interrogated in such a manner, a mark may be provided as a certified image level and/or certified video diagnostic level dependent on whether the medical professional has identified a condition present in the medical test images and/or videos correctly or incorrectly.

It will be appreciated also that the quality of the sound interface 19 of the diagnostic terminal 10 may be tested. This enables a suitability assessment response to be generated in respect of heart murmur tests, or breathing tests, performed at the operator terminal 20 using an electronic stethoscope. Headphone interfaces and loudspeaker interfaces of the diagnostic terminal 10 may have differing audio responses, and the hearing ability of a user of the diagnostic terminal 10 may vary with age.

Therefore, the diagnostic terminal 10 may hold in its memory a number of audio tests, which optionally may be encrypted, providing sounds of various heart murmur conditions or breathing conditions of various levels of severity. Alternatively, the operator terminal may transmit a number of audio tests to the diagnostic terminal 10. It is more practical to transmit sound tests to the diagnostic terminal 10 because sound typically has a lower bandwidth than video or high-resolution images, for example.

Optionally, the diagnostic terminal 10 further comprises a sound interface 19. The suitability assessment response data comprises the results of a suitability test executed by the diagnostic terminal comprising the broadcast of a test sound from the sound interface 19, and the selection of a user of a diagnostic condition which the sound represents.

It will be appreciated that the tests discussed previously result in first, second, third, fourth, and fifth suitability assessment response data being generated. Accordingly, it is possible to generate first, second, third, fourth, and fifth suitability certificate data based on the suitability assessment response data, respectively.

Optionally, the first suitability assessment response data comprises a contrast response curve.

Optionally, the second suitability assessment response data comprises a certified graphical test, or the certified video selection.

Optionally, the third suitability assessment response data comprises certified display configuration data.

Optionally, the fourth suitability assessment response data comprises a certified image level and/or the certified video diagnostic level.

Optionally, the fifth suitability assessment response data comprises a certified selection of a diagnostic condition represented by a sound.

According to embodiments of the first aspect, the suitability assessment response data may comprise a composite of any one of the first, second, third, fourth, and fifth suitability assessment response data.

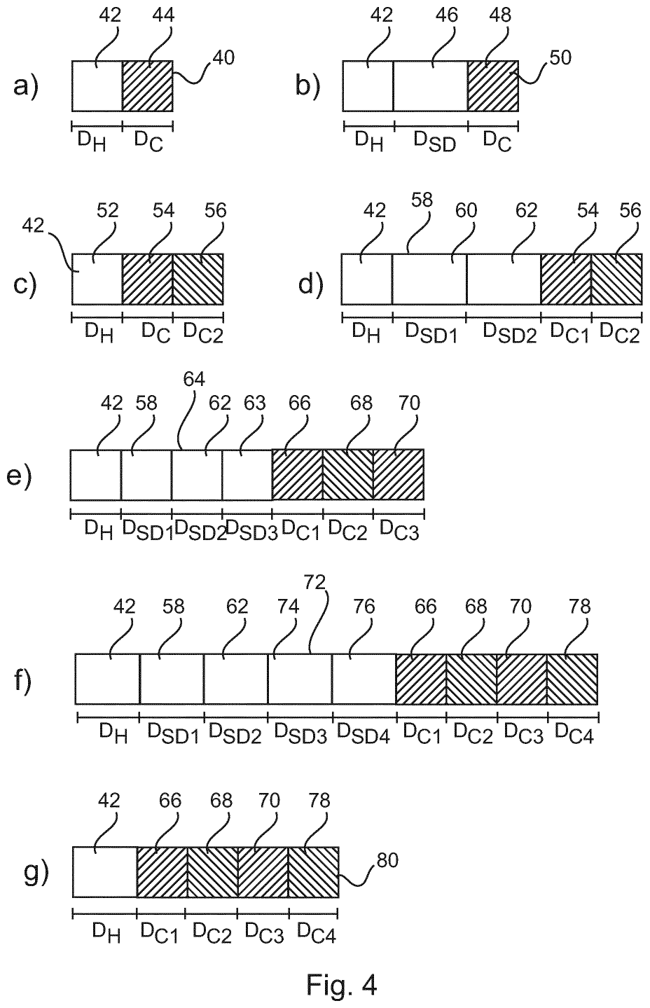

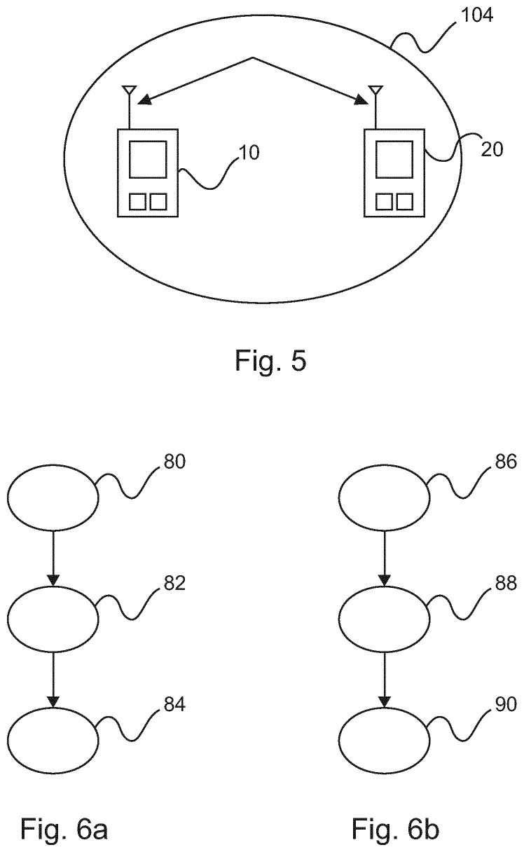

Examples of data structures communicated by the first data communication interface 12 of the diagnostic terminal are illustrated in FIGS. 4a)-4g).

FIG. 4a) illustrates a simple case where a data packet 40 is transmitted comprising a data header, DH 42 and a suitability certificate data payload, Dc 44. The data header comprises the usual addressing and networking parameters necessary to enable the data packet to be transmitted from the diagnostic terminal 10 to the operator terminal 20 over a data communications network. Therefore, at the conclusion of a test, the user, or the first display 14 of the diagnostic terminal 10 is classified and certified according to the suitability certificate data 44, and the suitability certificate data 44 is transmitted to an operator terminal 20. Thus, a low bandwidth capability assessment of the diagnostic terminal 10 may be provided.

FIG. 4b) shows an alternative, in which a packet 48 is transmitted comprising the data header 42, a suitability assessment response data payload 46, and a suitability certificate data payload 50. In this case, the same suitability certificate data 48 as generated at the diagnostic terminal 10 is transmitted as in the example of FIG. 4a), but the suitability assessment response data used to generate the suitability certificate data is also transmitted to the operator terminal 20. This enables the suitability assessment response data 48 to be used to verify the content of the suitability certificate data 50 at the operator terminal, enhancing security. As discussed previously, many combinations of suitability assessment response data may be used according to this technique.

FIG. 4c) shows a data packet comprising the data header 42, first suitability certificate data 54 and second suitability certificate data 56. Thus, the certified outcomes of two tests may be transmitted from the diagnostic terminal 10 to the operator terminal 20. FIG. 4d) shows a packet 58 comprising data header 42 to enable the packet to be transmitted from the diagnostic terminal 10 to the operator terminal 20, and first 60 and second 62 suitability assessment response data payloads, with the first 54 and second 56 suitability certificate data payloads appended to the packet.

FIG. 4e) shows a data packet 64, in which the data header 42 is provided with a first 58, second 62, and third 63 suitability assessment response data portions, with accompanying respective first 66, second 68, and third 70 suitability certificate data payloads.

FIG. 40 shows a large data packet 72 comprising the data header 42, the first 58, second 62, third 63, and fourth 76 suitability assessment response data portions, to which are appended the first 66, second 68, third 70, and fourth 78 suitability certificate data. Of course, alternatively, the combination of four suitability certificate data payloads can be transmitted on their own.

FIG. 4g) shows a packet 80 in which the data header 42 is provided with first 66, second 68, third 70, and fourth 78 suitability certificate data payloads.

Accordingly, it will be appreciated that a large combination of the tests described previously may be transmitted from the diagnostic terminal 10 to the operator terminal 20. Alternatively, a subset of the tests, or one test can be transmitted. Alternatively, data referring to only the suitability certificate data may be transferred. Alternatively, the suitability certificate data may be transferred in combination with the suitability assessment response data used to generate the suitability certificate data. Alternatively, for some tests, the suitability certificate data may be transmitted with the suitability assessment response data, and other tests may be transmitted only with the suitability certificate data.

Optionally, the transmitted suitability certificate data and/or the suitability assessment response data are encrypted to improve data security.

Optionally, the suitability certificate data and suitability assessment response data are cryptographically authenticated between the diagnostic terminal 10 and the operator terminal 20 to enhance trust between users of the terminals.

Therefore, the electronic certificate may, in an embodiment, use encryption. The software stored in the diagnostic terminal 10 may be encrypted to prevent access of a diagnostician to the test software.

Optionally, the test software may be loaded and initialized by a diagnostician. After the completion of a generic test, a generic certificate is created reflecting the test results, guaranteeing a certain test result. For example, this certificate may be valid only on the display device it was created on. The certificate may, for example, be valid for a limited time only. Optionally, the test has to be repeated on expiry. Optionally, the certificate can be transferred along with a diagnosis or the advice given to the diagnostician. Optionally, the certificate may be analyzed by the operator terminal at the patient location, and used either by the software or by the user operating it to assess the quality of the diagnosis according to it. According to an embodiment of the first aspect, the diagnostic terminal 10 is provided as previously described, wherein the suitability certificate data comprises, or is, an encrypted data portion encrypted by the first processing unit 18.

In other words, once a result has been generated from the suitability assessment response data, the suitability certificate data is generated, and encrypted according to one of a number of encryption algorithms.

Optionally, the suitability certificate data may be encrypted according to a public key encryption concept, in which the doctor's registration number forms part of the public key.

Optionally, the suitability certificate data may be encrypted according to a private encryption method, in which the key is transmitted via an alternative channel to the diagnostician, such as over the phone. The suitability certificate data may be encrypted, but portions of the suitability assessment response data may remain unencrypted. Optionally, the entire data portion transmitted by the first processing unit 18 may be encrypted.

Alternative suitability certificates from different tests may be encrypted using different encryption keys or techniques.

Optionally, the diagnostic terminal 10 as previously described is provided wherein the suitability certificate data comprises an authenticated time-stamp representing a time point when the suitability assessment response data was generated.

Accordingly, it is possible for an operator terminal 20 to assess how long ago a suitability test was performed, and to issue a suitability test challenge to the diagnostic terminal 10 if the authenticated time-stamp indicates that the suitability certificate data is too old.

Optionally, the diagnostic terminal 10 may use the authenticated time-stamp on the suitability certificate data to itself automatically reschedule a suitability assessment challenge proactively.

Optionally, the first processing unit 18 is further configured to calculate a time-delay of an authenticated time-stamp of the suitability certificate data from a current time point, and wherein, if the time lapse is greater than a certification time threshold, the first processing unit 18 is configured to repeat the generation of the suitability certificate data at the diagnostic terminal.

Accordingly, it is possible to assess the age of a suitability certificate. Until now, the discussion has centered around the generation of challenges and the use of tests to verify the ability of a diagnostic terminal and/or the user. This has not concerned a real diagnosis.

Optionally, however, it may be attractive to include the relevant suitability certification with the response of a real diagnosis, to improve trust in a real diagnostic exchange between an operator terminal and a diagnostic terminal.

Optionally, the first processing unit 18 is further configured to receive (i) diagnosis request data and (ii) pre-diagnostic data via the first data communication interface 12 from an operator terminal 20, to display the diagnosis request data and the pre-diagnostic data to a medical professional, to receive diagnosis data from the medical professional, to generate certified diagnosis data by combining the suitability certificate data with the diagnosis data and to transmit the certified diagnosis data to an operator terminal via the first data communication interface 12.

In other words, pre-diagnosis request data, such as a text string stating “is this an example of lung cancer?”, and pre-diagnostic data, such as a chest X-ray taken by equipment connected to the operator terminal 20, or provided to the operator terminal 20, may be communicated to the diagnostic terminal 10. After performing a diagnostic challenge, or using the results of a previous diagnostic challenge, the diagnostician using diagnostic terminal 10 may respond to the diagnosis request data by incorporating the suitability certificate data into the pre-diagnostic data with the diagnostic response. For example, the diagnostician would provide, in textual form, their comments in alphanumeric format. The diagnostic terminal 10 would incorporate the alphanumeric comments into the response. In the pre-diagnostic image itself, the diagnostic terminal 20 would insert a visual certification element, such as a barcode, a QR code, or a text code in alphanumerics, giving the local caregiver at the operator terminal 20 an instant impression that the diagnostician was qualified to give such a diagnosis with such an image.

It will be appreciated that this development lies in the area of collaborative medical care. Optionally, the test software is an application that can execute on all common smartphones and tablets, or laptops and computers. The suitability assessment certificates are optionally provided according to a common standard in order to work in a heterogeneous environment.

The client system (operator terminal 20) can be used at the location where the image acquisition is done, and where support needs to be given to a patient. The client system (operator terminal 20) could, potentially, be part of the imaging equipment itself, such as a smart X-ray equipment, or a smart ultrasound equipment. This could provide access to a peer group of specialists. Alternatively, it could be a downloadable application independent of the acquisition system.

Optionally, the results of a particular device test may be used to optimize a display of the images on that particular device. Image post-processing can be optimized to achieve the best possible visual quality given at that device and observer. Any imaging modality has its own post-processing method, where a number of parameters can be set according to different imaging display properties. These could be adjusted based on the quality test proposed here.

According to a third aspect, an operator terminal 20 is provided for acquiring suitability assessment response data and/or suitability certificate data from a user of the diagnostic terminal. The operator terminal 20 comprises:

a second data communication interface 22;

a second display 24;

a second input interface 26; and

a second processing unit 28.

The second processing unit 28 is configured to receive suitability certificate data based on a suitability assessment result generated at the remote diagnostic terminal from the second data communication interface, to analyze the suitability certificate to provide a diagnostic terminal suitability result, and to display the suitability certificate, and/or the diagnostic terminal suitability result to a user of the operator terminal using the second display 24.

FIG. 1b) illustrates an example of an operator terminal 20.

Optionally, the second processing unit 28 of the operator terminal 20 is further configured to transmit suitability assessment challenge request data to a remote diagnostic terminal via the second data communication interface 22, and to instruct the remote diagnostic terminal to generate suitability certificate data and/or suitability assessment response data.

Optionally, the second processing unit 28 of the operator terminal 20 is further configured to analyze the suitability certificate data by identifying an encrypted data portion of the suitability certificate, decrypting the encrypted data portion of the suitability certificate, generating a diagnostic trust metric based on the decrypted result of the encrypted data portion of the suitability certificate, and displaying the diagnostic trust metric to a user of the operator terminal using the second display 24.

Optionally, the second processing unit 28 of the operator terminal 20 is further configured to analyze the suitability certificate data by extracting results of one, or a combination of, a diagnostic terminal contrast response, a graphical test or a video selection feedback result, certified display configuration data, or a certified image level and/or a certified video diagnostic level, to compare at least the results of one, or a combination of, a diagnostic terminal contrast response, a graphical test or a video selection feedback result, certified display configuration data, or a certified image level and/or a certified video diagnostic level with standard values, and to generate a diagnostic terminal test threshold, and wherein the second processing unit is configured to display the diagnostic terminal test threshold to a user on the second display 24.

Optionally, the second processing unit 28 of the operator terminal 20 is further configured to extract, from the suitability certificate data, an authenticated time-stamp representing the time point that the suitability assessment challenge had been performed, and to display, to a user of the operator terminal, the time point that the suitability assessment challenge had been performed using the second display 24.

Optionally, the second processing unit 28 of the operator terminal 20 is further configured to transmit to a diagnostic terminal (i) a diagnosis request and (ii) pre-diagnostic information via the second data communication interface 22, to receive, from the diagnostic terminal via the second data communication interface, certified diagnosis data comprising diagnosis data and a suitability certificate, and to display, to a user of the operator terminal, the diagnosis data and the suitability certificate using the second display 24.

According to a fifth aspect, there is provided a distributed medical information assurance system 104 comprising:

a diagnostic terminal 10 according to the first aspect or its optional embodiments;

an operator terminal 20 according to the third aspect or one of its optional embodiments;

wherein the diagnostic terminal 10 and the operator terminal 20 are configured to communicate suitability certificate data and/or suitability assessment response data.

Optionally, the distributed medical information assurance system 104 is implemented using a GSM, 3G, 4G, or 5G telephone network with data and/or internet data communication capabilities.

Optionally, the distributed medical information assurance system 104 is implemented using a satellite communications network with data and/or internet data communication capabilities.

Optionally, the distributed medical information assurance system 104 is implemented using a wired communications network using Digital Subscriber Line (DSL), for example.

According to a second aspect, a method for operating a diagnostic terminal to provide suitability certificate data of a user of the diagnostic terminal, to an operator terminal, is provided, comprising:
a) generating (80) suitability assessment response data based on a suitability test executed by the diagnostic terminal and/or by the user of the diagnostic terminal;
b) generating (82) suitability certificate data based on the suitability assessment response data generated by the diagnostic terminal; and
c) transmitting (84) the suitability certificate data and/or the suitability assessment response data to an operator terminal via a data communication interface.

Optionally, the diagnostic terminal method further comprises:
a1) receiving suitability assessment challenge request data transmitted to the diagnostic terminal by a remotely located operator terminal;

wherein the suitability assessment response data is generated based on the requirements of the suitability assessment challenge request data; and

wherein, in step c), the suitability certificate data and/or the suitability assessment response data is transmitted to the operator terminal, as a response to the suitability assessment challenge request data.

Optionally, generating the suitability assessment response data at the diagnostic terminal further comprises:
a2) interrogating the diagnostic terminal to derive a display contrast characteristic of a display of the diagnostic terminal.

Optionally, generating the suitability assessment response data at the diagnostic terminal further comprises:
a3) displaying a contrast test sequence on a display of the diagnostic terminal;
a4) recording the contrast test sequence generated by the display of the diagnostic terminal using a camera of the diagnostic terminal, wherein the camera and the display of the diagnostic terminal are arranged to face a mirror simultaneously;
a5) generating the suitability assessment response data by comparing the recorded contrast test sequence to a set of standard contrast test sequence results; and

wherein the suitability assessment response data comprises diagnostic terminal display contrast data.

Optionally, generating the suitability assessment response data at the diagnostic terminal further comprises:
a6) displaying, on a display of the diagnostic terminal, a certified graphical test image or certified video test sequence;
a7) receiving, via an input of the diagnostic terminal, a certified graphical test or a certified video selection as a user input command;
a8) generating the suitability assessment response data by comparing the certified graphical test, or the certified video selection, to a set of graphical test or a set of video selection feedback results; and

wherein the suitability assessment response data comprises the certified graphical test or the certified video selection.

Optionally, generating the suitability assessment response data at the diagnostic terminal further comprises:
a9) commanding the diagnostic terminal to yield display configuration information;
a10) verifying the display configuration information; and
a11) generating the suitability assessment response data by certifying the verified display configuration data; and

wherein the suitability assessment response data comprises the certified display configuration data.

Optionally, generating the suitability assessment response data at the diagnostic terminal further comprises:
a12) displaying, on a display of the diagnostic terminal, a plurality of medical test images and/or videos;
a13) for each of the displayed images and/or videos in the plurality of medical test images and/or videos, presenting a challenge input prompt to a user of the diagnostic terminal and obtaining a set of challenge answers;
a14) analyzing the set of challenge answers and generating an image and/or video diagnostic capability level;
a15) generating the suitability assessment response data by certifying the image and/or video diagnostic capability level; and

wherein the suitability assessment response comprises the certified image level and/or the certified video diagnostic level.

Optionally, in the method of the third aspect, the suitability certificate data

comprises, or is, an encrypted data portion encrypted at the diagnostic terminal.

Optionally, the suitability certificate data comprises an authenticated time-stamp representing a time point when the suitability assessment response data was generated. Optionally, the method of the third aspect further comprises:
a16) calculating a time-delay of an authenticated time-stamp of the suitability certificate data from a current time point, and
a17) if the time lapse is greater than a certification time threshold, repeating the generation of the suitability certificate data at the diagnostic terminal.

Optionally, the method of the third aspect further comprises:
a18) receiving (i) diagnosis request data and (ii) pre-diagnostic data from an operator terminal;
a19) displaying the diagnosis request data and the pre-diagnostic data to a medical professional;
a20) receiving diagnosis data from the medical professional;
a21) generating certified diagnosis data by combining the suitability certificate data with the diagnosis data;
a22) transmitting the certified diagnosis data to an operator terminal.

According to a fourth aspect, there is provided a method of operating an operator terminal to acquire suitability assessment response data and/or suitability certificate data of a diagnostic terminal, and/or a user of the diagnostic terminal. The method comprises:
b1) receiving (86) suitability certificate data based on a suitability assessment result generated at the remote diagnostic terminal;
b2) analyzing (88) the suitability certificate to provide the diagnostic terminal suitability result; and
b3) displaying (90) the suitability certificate, and/or the diagnostic terminal suitability result to a user of the operator terminal.

Optionally, the method of operating the operator terminal comprises:
b4) transmitting suitability assessment challenge request data to a remote diagnostic terminal, instructing the remote diagnostic terminal to generate suitability certificate data and/or suitability assessment response data.

Optionally, the method of operating the operator terminal comprises: analyzing the suitability certificate data by:
b5) identifying an encrypted data portion of the suitability certificate;
b6) decrypting the encrypted data portion of the suitability certificate;
b7) generating a diagnostic trust metric based on the decrypted result of the encrypted data portion of the suitability certificate; and
b8) displaying the diagnostic trust metric to a user of the operator terminal.

Optionally, the method of operating the operator terminal comprises: analyzing the suitability certificate data by:
b9) extracting, at the operator terminal, results of one, or a combination of, a diagnostic terminal contrast response, a graphical test or a video selection feedback result, certified display configuration data, or a certified image level and/or a certified video diagnostic level;
b10) comparing at least the results of one, or a combination of, a diagnostic terminal contrast response, a graphical test or a video selection feedback result, certified display configuration data, or a certified image level and/or a certified video diagnostic level with standard values, to generate a diagnostic terminal test threshold; and
b11) displaying the diagnostic terminal test threshold to a user.

Optionally, the method of operating the operator terminal comprises: analyzing the suitability certificate data by:
b12) extracting, from the suitability certificate data, an authenticated time-stamp representing the time point that the suitability assessment challenge had been performed; and
b13) displaying, to a user of the operator terminal, the time point that the suitability assessment challenge had been performed.

Optionally, the method of operating the operator terminal comprises:
b14) transmitting to a diagnostic terminal (i) a diagnosis request and (ii) pre-diagnostic information;
b15) receiving, from the diagnostic terminal, certified diagnosis data comprising diagnosis data and a suitability certificate;
b16) displaying, to a user of the operator terminal, the diagnosis data and the suitability certificate.

According to a sixth aspect, there is provided a distributed medical information assurance system method, comprising:
p) generating (92) suitability assessment response data based on a suitability test executed by the diagnostic terminal and/or by the user of the diagnostic terminal;
q) generating (94) suitability certificate data based on the suitability assessment response data generated by the diagnostic terminal;
r) transmitting (96) the suitability certificate data and/or the suitability assessment response data to an operator terminal via data communication interface;
s) receiving (98) suitability certificate data based on suitability assessment result generated of the remote diagnostic terminal;
t) analyzing (100) the suitability certificate to provide a diagnostic terminal suitability result; and
u) displaying (102) the suitability certificate, and/or the diagnostic terminal suitability result to a user of the operator terminal.

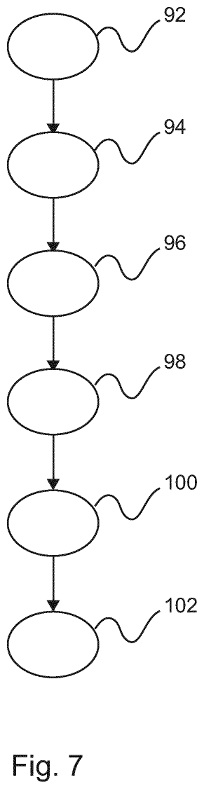

FIG. 7 illustrates a distributed medical information assurance system method in accordance with the sixth aspect.

According to a seventh aspect there is provided a computer program element for controlling the diagnostic method according to the first aspect or its embodiments, and/or an operator terminal according to the third aspect and its embodiments, which, when the computer program element is executed by a processing unit, is adapted to perform the method of the second or fourth aspect, respectively.

According to an eighth aspect of the invention, there are provided computer-readable media having stored the computer program elements of the seventh aspect.

A computer program element might therefore be stored on a computer unit, which might also be part of an embodiment of the present invention. This computing unit may be adapted to perform or induce performance of the steps of the method described above.

Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both the computer program that has the invention installed from the beginning, and a computer program that by means of an update turns an existing program into a program that uses the invention. A computer program may be stored and/or distributed on a suitable medium, such as an optical storage media or a solid state medium supplied together with, or as a part of other hardware, but may also be distributed in other forms, such as via the Internet or other wired or wireless telecommunication systems.

However, the program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It should to be noted that embodiments of the invention are described with reference to different subject-matters. In particular, some embodiments are described with reference to method-type claims, whereas other embodiments are described with reference to the device-type claims. However, a person skilled in the art will gather from the above, and the following description, that unless otherwise notified, in addition to any combination of features belonging to one type of subject-matter, also any other combination between features relating to different subject-matters is considered to be disclosed with this application.

All features can be combined to provide a synergetic effect that is more than the simple summation of the features.

While the invention has been illustrated and described in detail in the drawings and foregoing description, such illustration and description are to be considered illustrative or exemplary, and not restrictive. The invention is not limited to the disclosed embodiments.

Other variations to the disclosed embodiments can be understood, and effected by those skilled in the art in practicing the claimed invention, from a study of the drawings, the disclosure, and the dependent claims.

In the claims, the word “comprising” does not exclude other elements or steps, and the indefinite article “a” or “an” does not exclude a plurality. A single processor, or other unit, may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. A diagnostic terminal for providing suitability certificate data of a a diagnostic terminal to an operator terminal, comprising:
a data communication interface configured to transmit data to and from the diagnostic terminal;
a display configured to display the data;
an interface configured to enable a control of the displayed data; and
a processor configured to:
generate suitability assessment response data based on a suitability test executed by the diagnostic terminal;
generate suitability certificate data based on the suitability assessment response data: and
transmit at least one of the suitability certificate data and the suitability assessment response data to the operator terminal via the data communication interface.

2. The diagnostic terminal of claim 1, wherein the processor is further configured to receive suitability assessment challenge request data transmitted to the diagnostic terminal by the operator terminal, wherein the suitability assessment response data is generated based on requirements of the suitability assessment challenge request data; and wherein at least one of the suitability certificate data and the suitability assessment response data is transmitted to the operator terminal via the data communication interface as a response to the suitability assessment challenge request data.

3. The diagnostic terminal of claim 1, further comprising:
a camera, wherein the processor is configured to generate the suitability assessment response data by displaying a contrast test sequence, record the contrast test sequence using the camera, wherein the camera and the display are arranged to face a mirror simultaneously, and generate the suitability assessment response data by comparing the recorded contrast test sequence to a set of standard contrast test sequence results, and wherein the suitability assessment response data comprises diagnostic terminal display contrast data of the display.

4. The diagnostic terminal according to claim 1, wherein the processor is configured to generate the suitability assessment response data by displaying a certified graphical test image or certified video test sequence, receiving a certified graphical test or a certified video selection generating the suitability assessment response data by comparing the certified graphical test or the certified video selection to a set of graphical test or a set of video selection feedback results; and wherein the suitability assessment response data comprises the certified graphical test or the certified video selection of the first display.

5. The diagnostic terminal according to claim 1, wherein the processor is configured to generate the suitability assessment response data by commanding the diagnostic terminal to yield display configuration information of the display, verify the display configuration information, and generate the suitability assessment response data by certifying the verified display configuration data, and wherein the suitability assessment response data comprises the certified display configuration data.

6. The diagnostic terminal according to claim 1, wherein the processor is configured to generate the suitability assessment response data by displaying a plurality of medical test images and/or videos, and for each of the displayed images and/or videos in the plurality of medical test images and/or videos, the processor is configured to present a challenge input prompt on the display and obtain a set of challenge answers via the input interface, analyze the set of challenge answers, generate an image and/or video diagnostic capability level, and generate the suitability assessment response data by certifying the image and/or video diagnostic capability level, wherein the suitability assessment response comprises the certified image level and/or the certified video diagnostic level.

7. The diagnostic terminal according to claim 1, wherein the suitability certificate data comprises an encrypted data portion encrypted by the processor.

8. The diagnostic terminal according to claim 1, wherein the suitability certificate data comprises an authenticated time-stamp representing a time point when the suitability assessment response data is generated.

9. The diagnostic terminal according to claim 1, wherein the processor is further configured to calculate a time-delay of an authenticated time-stamp of the suitability certificate data from a current time point, and wherein if the time lapse is greater than a certification time threshold processor is configured to repeat the generation of the suitability certificate data.

10. The diagnostic terminal according to claim 1, wherein the processor is further configured to receive diagnosis request data and pre-diagnostic data via the data communication interface from the operator terminal, display the diagnosis request data and the pre-diagnostic data to a medical professional, receive diagnosis data from the medical professional, generate certified diagnosis data by combining the suitability certificate data with the diagnosis data, and transmit the certified diagnosis data to the operator terminal via the data communication interface.

11-16. (canceled)

17. A distributed medical information assurance system, comprising:
a diagnostic terminal for providing suitability certificate data of a diagnostic terminal to an operator terminal, comprising:
a data communication interface configured to transmit data to and from the diagnostic terminal;
a display configured to display the data;
an interface configured to enable the use to control the displayed data; and
a processor configured to:
generate suitability assessment response data based on a suitability test executed by the diagnostic terminal;
generate suitability certificate data based on the suitability assessment response data; and
transmit at least one of the suitability certificate data and the suitability assessment response data to the operator terminal via the data communication interface;
an operator terminal for acquiring at least one of the suitability assessment response data and the suitability certificate data from the diagnostic terminal, wherein the diagnostic terminal and the operator terminal are configured to communicate at least one of the suitability certificate data and the suitability assessment response data.

18. A distributed medical information assurance method, comprising:
generating suitability assessment response data based on a suitability test executed by diagnostic terminal;
generating suitability certificate data based on the suitability assessment response data;
transmitting at least one of the suitability certificate data and the suitability assessment response data to an operator terminal via a data communication interface;
receiving the suitability certificate data based on a suitability assessment result of the diagnostic terminal;
analyzing the suitability certificate data to provide a diagnostic terminal suitability result; and
displaying at least one of the suitability certificate data and the diagnostic terminal suitability result to the operator terminal.

19. A method for operating a diagnostic terminal to provide suitability certificate data of the diagnostic terminal to an operator terminal, comprising:
generating a suitability assessment response data based on a suitability test executed by the diagnostic terminal;
generating suitability certificate data based on the suitability assessment response data; and
generating at least one of the suitability certificate data and the suitability assessment response data to an operator terminal via a data communication interface of the diagnostic terminal.

20-22. (canceled)

23. A non-transitory computer-readable medium having one or more executable instructions stored thereon, which, when executed by a processor, cause the processor to perform a method for operating a diagnostic terminal to provide suitability certificate data of the diagnostic terminal to an operator terminal, the method comprising:
generating a suitability assessment response data based on a suitability test executed by the diagnostic terminal;
generating suitability certificate data based on the suitability assessment response data; and
generating at least one of the suitability certificate data and the suitability assessment response data to an operator terminal via a data communication interface of the diagnostic terminal.

24. The diagnostic terminal according to claim 1, wherein the suitability certificate data comprises a data structure representing an authenticated assessment indicative of the ability of the diagnostic terminal to form a valid opinion about a radiographic image.
